(19) United States
(12) Patent Application Publication
(10) Pub. No.: US 2024/0245653 A1
(43) Pub. Date: Jul. 25, 2024

(54) PHARMACEUTICAL FORMULATIONS

(71) Applicant: SPEPHARM AG, Luzern (CH)

(72) Inventors: Samuel SMITH, Harefield, Uxbridge (GB); Luke EDWARDS, Harefield, Uxbridge (GB); Susanna SAUNDERS, Harefield, Uxbridge (GB); Lorenzo MENCUCCINI, Ferentino (IT)

(73) Assignee: SPEPHARM AG, Luzern (CH)

(21) Appl. No.: 18/559,213

(22) PCT Filed: May 6, 2022
(86) PCT No.: PCT/EP2022/062307

§ 371 Date: Nov. 6, 2023

(30) Foreign Application Priority Data
May 6, 2021 (GB) 2106497.7
Sep. 8, 2021 (GB) 2112820.2

(51) Int. Cl. A61K 31/4178; A61K 9/08; A61K 9/19; A61K 47/10; A61K 47/40

(52) U.S. Cl. CPC A61K 31/4178; A61K 9/08; A61K 9/19; A61K 47/10; A61K 47/40

(57) ABSTRACT

A formulation comprising dantrolene, or a pharmaceutically acceptable salt thereof, and a cyclodextrin in a molar ratio of 1:3 to 1:12 and also comprising polyethylene glycol (PEG) with an average molecular weight in the range 1500 to 6000.

### FIELD OF THE INVENTION

The present invention concerns improved formulations of dantrolene. More particularly, but not exclusively, this invention concerns improved dantrolene formulations with, amongst other advantages, improved solubility.

### BACKGROUND OF THE INVENTION

Dantrolene (1-({[5-(4-Nitrophenyl)furan-2-yl]methylidene}amino)imidazole-idine-2,4-dione), commonly used in its sodium salt form dantrolene sodium, is a skeletal muscle relaxant used for relieving chronic severe spasticity and malignant hyperthermia. Malignant hyperthermia is a genetic sensitivity of skeletal muscles to volatile anaesthetics and depolarizing neuromuscular-blocking drugs used for general anaesthesia. In susceptible individuals, these drugs can induce a drastic and uncontrolled increase in skeletal oxidative metabolism, which overwhelms the body's capacity to supply oxygen, remove carbon dioxide and regulate body temperature, eventually leading to circulatory collapse and death if not treated quickly.

Dantrolene exerts its effect on skeletal muscles by impacting calcium efflux and lessening the excitation-contraction coupling in muscle cells, leading to a reduction in the force of the contractile process.

Dantrolene (as dantrolene sodium) is also under development for the treatment of other conditions, including acute radiation syndrome in hematopoietic syndrome patients exposed to high doses of radiation, psychostimulant drug-induced toxicity (MDMA and Methamphetamine Intoxication), exertional heat stroke, cerebral concussion and other forms of traumatic brain injury, nerve-agent induced brain damage and coronavirus infection (COVID-19).

Although dantrolene is a good skeletal muscle relaxant, its pharmacological effects are limited because it is poorly soluble in water. This poor solubility leads to difficulties in the preparation of solutions suitable for intravenous administration and the necessity to administer large volumes of solution in a short space of time order to deliver an efficacious dose. Poor solubility also impacts the pharmaceutically-acceptable salts of dantrolene, which, when in solution, gradually precipitate in the form of the free acid, making the solutions unacceptable for injection.

Cyclodextrins are cyclic oligosaccharides, which consist of a ring of glucose subunits joined by α-1,4 glycosidic bonds. They have various uses within the food and pharmaceutical industries, including in the context of drug delivery and stabilisation.

WO 2018/146187 describes pharmaceutical formulations comprising a complex of dantrolene or its salts and a cyclodextrin. However, the authors of WO 2018/146,187 found that the addition of further compounds to the formulations reduced the solubility of the dantrolene.

There therefore remains a need to provide formulations of dantrolene and its salts which have improved solubility in aqueous solvents, thus facilitating the administration of the drug to patients in need thereof.

### SUMMARY OF THE INVENTION

The present invention provides, according to a first aspect, a formulation comprising dantrolene, or a pharmaceutically acceptable salt thereof, and a cyclodextrin in a molar ratio of 1:3 to 1:12 and also comprising polyethylene glycol (PEG) with an average molecular weight in the range 1500 to 6000.

According to a second aspect of the invention, there is provided a dry formulation prepared by drying a liquid formulation as described herein.

According to a third aspect of the invention, there is provided a dry formulation comprising 100-130 mg dantrolene sodium, 3000-4000 mg 2-hydroxypropyl-β-cyclodextrin and 350-450 mg PEG3350.

According to a fourth aspect of the invention, there is provided a liquid formulation prepared by dissolving a dry formulation as described herein in a pharmaceutically acceptable solvent.

According to a fifth aspect of the invention, there is provided an aqueous solution comprising dantrolene or a pharmaceutically acceptable salt thereof and a cyclodextrin in a molar ratio of 1:3 to 1:12 and also comprising PEG with an average molecular weight in the range 1500 to 6000.

According to a sixth aspect of the invention, there is provided an aqueous solution comprising: (i) 6 mg/mL dantrolene sodium hemiheptahydrate equivalent; (ii) 176.5 mg/mL 2-hydroxypropyl-β-cyclodextrin and (iii) 20 mg/ml PEG3350.

According to a seventh aspect of the invention, there is provided an aqueous solution comprising: (i) 5.3 mg/mL dantrolene sodium hemiheptahydrate equivalent; (ii) 156.2 mg/mL 2-hydroxypropyl-β-cyclodextrin and (iii) 17.7 mg/ml PEG3350.

According to an eighth aspect of the invention, there is provided a vial comprising: (i) 101 mg anhydrous dantrolene sodium (equivalent to 120 mg dantrolene sodium hemiheptahydrate); (ii) 3.530 g 2-hydroxypropyl-β-cyclodextrin and (iii) 400 mg PEG3350.

According to a ninth aspect of the invention, there is provided a formulation, an aqueous solution or a vial as described herein, for use as a medicament.

According to a tenth aspect of the invention, there is provided a formulation, an aqueous solution or a vial as described herein, for use in the treatment of malignant hyperthermia.

It will of course be appreciated that features described in relation to one aspect of the present invention may be incorporated into other aspects of the present invention. For example, the formulations of the invention may incorporate any of the features described with reference to the compositions and methods of the invention and vice versa.

### DETAILED DESCRIPTION

The present invention relates to a formulation comprising a pharmaceutically acceptable salt of dantrolene and a cyclodextrin in a molar ratio of 1:3 to 1:12 and also comprising PEG with an average molecular weight in the range 1500 to 6000.

The present invention is directed to an improved formulation of dantrolene. In particular, the present invention is based on the surprising finding that the inclusion of a cyclodextrin and a polyethylene glycol (PEG) in the recited molecular weight range results in a formulation that has good solubility of dantrolene and also has other beneficial properties in aqueous solution. The achievement of both of these properties is especially surprising. Previous disclosures have reported that, whilst a cyclodextrin may improve the solubility of dantrolene or a salt thereof, the inclusion of further components has a strongly adverse effect and negates the improved solubility. The formulations of the present invention simultaneously achieve good solubility and other beneficial properties in aqueous solution.

In particular, the inclusion of a PEG in the recited molecular weight range in the formulation of the invention reduces or prevents foaming when an aqueous solution of the formulation is agitated. This provides the further advantage of reducing the time required to make a solution as described herein “injection-ready”. Agitation is generally required when a formulation (which is generally a dry formulation) is admixed with aqueous solvent to convert it to liquid form before use. This is critical in the clinical situations in which dantrolene is generally used (e.g. the treatment of malignant hyperthermia), where rapidity of drug preparation and administration is key.

Accordingly, in another aspect of the invention, there is provided a use of PEG to reduce foaming in a formulation during agitation, wherein said formulation comprises: (i) dantrolene, or a pharmaceutically acceptable salt thereof, and (ii) a cyclodextrin; and wherein the solubility of the dantrolene in said formulation additionally comprising PEG is not reduced compared to the solubility of dantrolene in said formulation not comprising PEG. In a further aspect, there is provided a PEG for use in reducing foaming in a formulation during agitation, wherein said formulation comprises: (i) dantrolene, or a pharmaceutically acceptable salt thereof, and (ii) a cyclodextrin; and wherein the solubility of the dantrolene in said formulation additionally comprising PEG is not reduced compared to the solubility of dantrolene in said formulation not comprising PEG. In a further aspect, there is provided a method for reducing foaming in a formulation during agitation, wherein said formulation comprises: (i) dantrolene, or a pharmaceutically acceptable salt thereof, and (ii) a cyclodextrin; wherein said method comprises adding PEG to said formulation, and wherein the solubility of the dantrolene in said formulation additionally comprising PEG is not reduced compared to the solubility of dantrolene in said formulation not comprising PEG. In another aspect, there is provided a method of preparing an aqueous formulation comprising the steps of: (i) providing a dry formulation comprising dantrolene, or a pharmaceutically acceptable salt thereof, a cyclodextrin and PEG; (ii) admixing said dry formulation with aqueous solvent and (iii) agitating said admixture to convert the dry formulation to an aqueous form, wherein the presence of the PEG reduces foaming while agitating said admixture when compared with an equivalent formulation not containing the PEG, and wherein the solubility of the dantrolene in said formulation comprising PEG is not reduced compared to the solubility of dantrolene in an equivalent formulation not containing the PEG. In the aspects of the invention described in this paragraph, the dantrolene, the cyclodextrin and the PEG may be defined as described elsewhere herein. Accordingly, in another aspect of the invention, there is provided a use of PEG having an average molecular weight in the range 1500 to 6000 to reduce foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, and wherein the solubility of the dantrolene in said formulation additionally comprising PEG is not reduced compared to the solubility of dantrolene in said formulation not comprising PEG. In a further aspect, there is provided a PEG having an average molecular weight in the range 1500 to 6000 for use in reducing foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, and wherein the solubility of the dantrolene in said formulation additionally comprising said PEG is not reduced compared to the solubility of dantrolene in said formulation not comprising PEG. In a further aspect, there is provided a method for reducing foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, wherein said method comprises adding PEG having an average molecular weight in the range 1500 to 6000 to said formulation and wherein the solubility of the dantrolene in said formulation additionally comprising said PEG is not reduced compared to the solubility of dantrolene in said formulation not comprising said PEG. In another aspect, there is provided a method of preparing an aqueous formulation comprising the steps of: (i) providing a dry formulation comprising (a) dantrolene, or a pharmaceutically acceptable salt thereof; (b) a cyclodextrin, wherein said dantrolene and said cyclodextrin are present in said formulation in a molar ratio of 1:3 to 1:12 and (c) PEG having an average molecular weight in the range 1500 to 6000; (ii) admixing said dry formulation with aqueous solvent and (iii) agitating said admixture to convert the dry formulation to an aqueous form, wherein the presence of the PEG reduces foaming while agitating said admixture when compared with an equivalent formulation not containing the PEG, and wherein the solubility of the dantrolene in said formulation comprising PEG is not reduced compared to the solubility of dantrolene in an equivalent formulation not containing the PEG. In the aspects of the invention described in this paragraph, the formulation, the dantrolene, the cyclodextrin and/or the PEG may be further defined as described elsewhere herein.

In another aspect of the invention, there is provided a use of PEG to reduce foaming in a formulation during agitation, wherein said formulation comprises: (i) dantrolene, or a pharmaceutically acceptable salt thereof, and (ii) a cyclodextrin; and wherein the solubility of the dantrolene in said formulation additionally comprising PEG is enhanced compared to the solubility of dantrolene in an equivalent formulation comprising the PEG, but not comprising the cyclodextrin. In a further aspect, there is provided a PEG for use in reducing foaming in a formulation during agitation, wherein said formulation comprises: (i) dantrolene, or a pharmaceutically acceptable salt thereof, and (ii) a cyclodextrin; and wherein the solubility of the dantrolene in said formulation additionally comprising PEG is enhanced compared to the solubility of dantrolene in an equivalent formulation comprising the PEG, but not comprising the cyclodextrin. In a further aspect, there is provided a method for reducing foaming in a formulation during agitation, wherein said formulation comprises: (i) dantrolene, or a pharmaceutically acceptable salt thereof, and (ii) a cyclodextrin; wherein said method comprises adding PEG to said formulation, and wherein the solubility of the dantrolene in said formulation additionally comprising PEG is enhanced compared to the solubility of dantrolene in an equivalent formulation comprising the PEG, but not comprising the cyclodextrin. In another aspect, there is provided a method of preparing an aqueous formulation comprising the steps of: (i) providing a dry formulation comprising dantrolene, or a pharmaceutically acceptable salt thereof, a cyclodextrin and PEG; (ii) admixing said dry formulation with aqueous solvent and (iii) agitating said admixture to convert the dry formulation to an aqueous form, wherein the presence of the PEG reduces foaming while agitating said admixture when compared with an equivalent formulation not containing the PEG, and wherein the solubility of the dantrolene in said formulation comprising PEG is enhanced compared to the solubility of the dantrolene in an equivalent formulation comprising the PEG, but not comprising the cyclodextrin. In the aspects of the invention described in this paragraph, the formulation, the dantrolene, the cyclodextrin and/or the PEG may be defined as described elsewhere herein. Accordingly, in another aspect of the invention, there is provided a use of PEG having an average molecular weight in the range 1500 to 6000 to reduce foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, and wherein the solubility of the dantrolene in said formulation additionally comprising said PEG is enhanced compared to the solubility of dantrolene in an equivalent formulation comprising the PEG, but not comprising the cyclodextrin. In a further aspect, there is provided a PEG having an average molecular weight in the range 1500 to 6000 for use in reducing foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, and wherein the solubility of the dantrolene in said formulation additionally comprising said PEG is enhanced compared to the solubility of dantrolene in an equivalent formulation comprising the PEG, but not comprising the cyclodextrin. In a further aspect, there is provided a method for reducing foaming in a formulation during agitation, wherein said formulation comprises: (i) dantrolene, or a pharmaceutically acceptable salt thereof, and (ii) a cyclodextrin; wherein said dantrolene and said cyclodextrin are present in said formulation in a molar ratio of 1:3 to 1:12, wherein said method comprises adding PEG having an average molecular weight in the range 1500 to 6000 to said formulation and wherein the solubility of the dantrolene in said formulation additionally comprising said PEG is enhanced compared to the solubility of dantrolene in an equivalent formulation comprising the PEG, but not comprising the cyclodextrin. In another aspect, there is provided a method of preparing an aqueous formulation comprising the steps of: (i) providing a dry formulation comprising (a) dantrolene, or a pharmaceutically acceptable salt thereof; (b) a cyclodextrin, wherein said dantrolene and said cyclodextrin are present in said formulation in a molar ratio of 1:3 to 1:12 and (c) PEG having an average molecular weight in the range 1500 to 6000; (ii) admixing said dry formulation with aqueous solvent and (iii) agitating said admixture to convert the dry formulation to an aqueous form, wherein the presence of the PEG reduces foaming while agitating said admixture when compared with an equivalent formulation not containing the PEG, and wherein the solubility of the dantrolene in said formulation comprising PEG is enhanced compared to the solubility of the dantrolene in an equivalent formulation comprising the PEG, but not comprising the cyclodextrin. In the aspects of the invention described in this paragraph, the formulation, the dantrolene, the cyclodextrin and/or the PEG may be further defined as described elsewhere herein. Enhanced solubility of dantrolene may be defined as a dantrolene solubility in a formulation as described herein, e.g. an aqueous formulation, of at least 0.3 mg/ml, at least 0.33 mg/ml, at least 0.5 mg/ml, at least 1 mg/ml, at least 1.5 mg/ml, at least 2 mg/ml, at least 2.5 mg/ml, at least 3 mg/ml, at least 3.5 mg/ml, at least 4 mg/ml at least 4.5 mg/ml, at least 5 mg/ml, at least 5.3 mg/ml or at least 5.5 mg/ml, for example about 6 mg/ml hemiheptahydrate equivalent. Enhanced solubility of dantrolene may be defined as a dantrolene solubility in a formulation as described herein, e.g. an aqueous formulation, of between 0.4 mg/ml and 10 mg/ml, between 0.5 mg/ml and 10 mg/ml, between 1 mg/ml and 10 mg/ml, between 1.5 mg/ml and 10 mg/ml, between 2 mg/ml and 10 mg/ml, between, 2.5 mg/ml and 10 mg/ml, between 3 mg/ml and 10 mg/ml, between 3.5 mg/ml and 10 mg/ml, between 4 mg/ml and 10 mg/ml, between 4.5 mg/ml and 10 mg/ml, between 5 mg/ml and 10 mg/ml, between 5.3 and 10 mg/ml, between 5.5 mg/ml and 10 mg/ml, between 0.5 mg/ml and 8 mg/ml, between 0.5 mg/ml and 7 mg/ml, between 0.5 mg/ml and 6 mg/ml, between 1 mg/ml and 7 mg/ml, between 1.5 and 7 mg/ml, between 2 mg/ml and 7 mg/ml, between 2.5 mg/ml and 7 mg/ml, between 3 mg/ml and 7 mg/ml or between 4 mg/ml and 6 mg/ml hemiheptahydrate equivalent.

Furthermore, the formulations of the present invention also permit the preparation of liquid formulations of dantrolene which reach a higher concentration of dantrolene and/or a lower reconstitution volume, thus further facilitating the rapid administration of the drug to a patient.

The inventors have also found that it is not necessary to include a pH adjusting or buffering component. The formulations described herein achieve the desired solubility and other beneficial effects without the need to include further components to influence the pH. The need for fewer components reduces clinical precautions that can be needed, simplifies the regulatory consideration of the product and also simplifies the manufacture and storage of the formulations.

Generally, for clinical use, the dantrolene product is supplied in a vial with instructions to add solvent immediately before use. The vial has a size suitable for the volume of solvent that is to be added. The lower reconstitution volume that can be achieved by the current formulations mean that smaller vials can be used, thus further reducing the hospital storage space required, and also reducing the cost of goods (CoGs).

In the formulation of active ingredients that are sparingly soluble or only slowly soluble, it is common to micronise the active ingredients. The formulations of the present invention allow the use of dantrolene with larger particle sizes than has generally been the case in the prior art. This also reduces the CoG of the drug product.

Dantrolene may exist in the form of the free acid which is represented by the following structural formula:

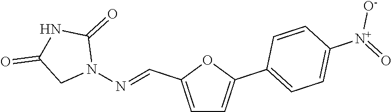 [SMILES: O=C1CN(/N=C/c2ccc(-c3ccc([N+](=O)[O-])cc3)o2)C(=O)N1]

More frequently, dantrolene is used in the form of a pharmaceutically acceptable salt, for example the sodium salt. Dantrolene sodium may be represented by the following structural formula:

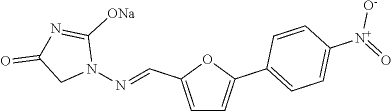

Formulations of dantrolene sodium exist in the art, for example DANTRIUM® IV 20 mg, as marketed in the UK by Norgine Pharmaceuticals Limited, Uxbridge, UK.

A pharmaceutically acceptable salt of dantrolene refers to a deprotonated form of dantrolene and a cationic counterion. In an embodiment of the invention, the cationic counterion is selected from the group of alkali metals, alkaline earth metals, ammonium, alkyl-ammonium, polyalkyl-ammonium, aryl-ammonium, substituted or unsubstituted quinolizinium and substituted or unsubstituted pyridinium.

In an embodiment of the invention, the pharmaceutically acceptable salt of dantrolene refers to a salt of dantrolene in which the cationic counterion to the dantrolene anion is preferably selected from the group consisting of sodium, potassium, ammonium, calcium, magnesium, the ammonium salts of physiologically acceptable amino compounds, especially selected from the group consisting of arginine, lysine, meglumine, tromethamine, choline, benzyltrimethylammonium, tetramethylammonium, N-methylpyridinium, tetrabutylammonium, 2-(2,3-dihydroxy-1-propylamino)-quinolizinium, quinolizinium, 2-carbonyl-1-methylpyridinium, 2,3-dimethyl-1-phenyl-4-trimethyl-ammonium-3-pyrazolin-5-one, dimethylammonium, 1,3-dimethylimidazolium and 2-(1-hydroxy-2-methyl)propyltri-methylammonium.

In an embodiment of the invention, the pharmaceutically acceptable salt of dantrolene refers to a salt of dantrolene in which the cationic counterion to the dantrolene anion is selected from the group consisting of sodium, potassium, ammonium, calcium and magnesium. In a preferred embodiment of the invention, the pharmaceutically acceptable salt of dantrolene refers to a salt of dantrolene in which the cationic counterion to the dantrolene anion is sodium, i.e. the sodium salt of dantrolene (also termed dantrolene sodium). In an especially preferred embodiment of the invention, the pharmaceutically acceptable salt of dantrolene is the hemiheptahydrate of the sodium salt, i.e. dantrolene sodium hemiheptahydrate.

The dantrolene active pharmaceutical ingredient (API), e.g. dantrolene sodium or dantrolene sodium hemiheptahydrate, as included in an embodiment of the invention may optionally be micronized to a particular particle size. In an embodiment, a formulation of the invention comprises dantrolene, e.g. dantrolene sodium, at a particle size of ≤70 μm, for example ≤63 μm or ≤40 μm. In an embodiment, a formulation of the invention comprises dantrolene, e.g. dantrolene sodium at a particle size of 0.2 to 70 μm, for example 5 to 70 μm, 10 to 70 μm, 25 to 70 μm or 35 to 70 μm. In an embodiment, a formulation of the invention comprises dantrolene, e.g. dantrolene sodium, at a particle size of 0.2 to 50 μm, for example 5 to 50 μm, 10 to 50 μm, 25 to 50 μm or 35 to 50 μm. Preferably, the dantrolene, e.g. dantrolene sodium, is present at a particle size of about 5 to about 40 μm. For example, the dantrolene, e.g. dantrolene sodium, is present at a particle size of about 40 μm.

Cyclodextrins are cyclic oligosaccharides formed from (α-1,4)-linked glucose subunits. Cyclodextrins possess a hydrophobic central cavity and a hydrophilic outer surface and due to their truncated cone or torus, cyclodextrins may interact with appropriately-sized molecules to give rise to the formation of inclusion complexes. In an embodiment of the invention, a cyclodextrin in a formulation as described herein may have between 5 and 30, between 5 and 20, between 5 and 15, between 5 and 10, between 5 and 8 or between 6 and 8 glucose subunits in the cyclic structure. Cyclodextrins which have 6, 7 or 8 glucose subunits in their cyclic structure are known as α (alpha)-cyclodextrin, β (beta)-cyclodextrin and γ (gamma)-cyclodextrin respectively. In an embodiment of the invention, a cyclodextrin in a formulation as described herein has 7 glucose subunits, i.e. β-cyclodextrin.

In an embodiment of the invention, the glucose subunits of a cyclodextrin in a formulation as described herein may be substituted. The substituents are selected independently from the group consisting of alkyl, hydroxyalkyl, carboxyalkyl, alkylcarbonyl, carboxyalkoxyalkyl, sulfoalkyl, alkylcarbonyloxyalkyl and alkoxycarbonylalkyl. For example, said cyclodextrins may be substituted with a C₁₋₈ alkyl group, a C₁₋₆ alkyl group, a C₁₋₄ alkyl group, a C₁₋₈ hydroxyalkyl group, a C₁₋₆ hydroxyalkyl group, a C₁₋₄ hydroxyalkyl group, a C₁₋₈ sulfoalkyl group, a C₁₋₆ sulfoalkyl group or a C₁₋₄ sulfoalkyl group. In another embodiment of the invention, a cyclodextrin in a formulation as described herein may be substituted with a C₁₋₈ alkyl group, a C₁₋₆ alkyl group or a C₁₋₄ alkyl group, wherein said alkyl group is itself substituted with a hydroxyl group or a sulfo group. In the foregoing, the term “alkyl” is to be understood to include both straight and branched-hydrocarbon radicals. A sulfo group refers to an —SO₃H moiety or a corresponding pharmaceutically-acceptable salt thereof. Preferred counterions are those which are defined herein as the counterion for the pharmaceutically acceptable salt of dantrolene.

A formulation according to an embodiment of the invention comprises a cyclodextrin which contains 5 to 10 glucose subunits, e.g. 6 to 8 glucose subunits, e.g. 7 glucose subunits, wherein said glucose subunits are optionally substituted with a C₁₋₈ alkyl group, which is itself optionally substituted with a hydroxyl group or a sulfo group. A formulation according to an embodiment of the invention comprises a cyclodextrin which contains 5 to 10 glucose subunits, e.g. 6 to 8 glucose subunits, e.g. 7 glucose subunits, wherein said glucose subunits are optionally substituted with a C₁₋₄ alkyl group, which is itself optionally substituted with a hydroxyl group or a sulfo group. A formulation according to an embodiment of the invention comprises a cyclodextrin which contains 5 to 10 glucose subunits, e.g. 6 to 8 glucose subunits, e.g. 7 glucose subunits, wherein said glucose subunits are optionally substituted with a methyl group, a C₁₋₄ hydroxyalkyl group or a C₁₋₄ sulfoalkyl group. A formulation according to an embodiment of the invention comprises a cyclodextrin which contains 5 to 10 glucose subunits, e.g. 6 to 8 glucose subunits, e.g. 7 glucose subunits, wherein said glucose subunits are optionally substituted with a C₂₋₄ hydroxyalkyl group. Specific examples of a C₂₋₄ hydroxyalkyl group include hydroxyethyl, hydroxypropyl, e.g. 2-hydroxypropyl, and hydroxybutyl. Accordingly, a formulation according to an embodiment of the invention comprises a cyclodextrin which contains 5 to 10 glucose subunits, e.g. 6 to 8 glucose subunits, e.g. 7 glucose subunits, wherein said glucose subunits are optionally substituted with a 2-hydroxypropyl group, e.g. 2-hydroxypropyl-β-cyclodextrin (HP-β-CD or HPBCD). Specific examples of 2-hydroxypropyl-β-cyclodextrins which may be used in a formulation of an embodiment of the invention are Cavitron™ W7 (Ashland, Inc.) or Kleptose® HPB (Roquette). A specific example of a sulfoalkyl group is a sulfobutyl group. A specific example of a sulfoalkyl-substituted cyclodextrin which may be used in a formulation of an embodiment of the invention is sulfobutyl ether-β-cyclodextrin (Captisol®, Ligand, San Diego, CA, USA) as described in EP2583668, the contents of which are hereby incorporated by reference. A specific example of a methyl-substituted cyclodextrin which may be used in a formulation of an embodiment of the invention is randomly methylated β-cyclodextrin (RM-β-CD or RMBCD) (Cavasol® W7 M, Wacker, or Kleptose® Crysmeb, Roquette).

A formulation according to an embodiment of the invention comprises a cyclodextrin represented by Formula I:

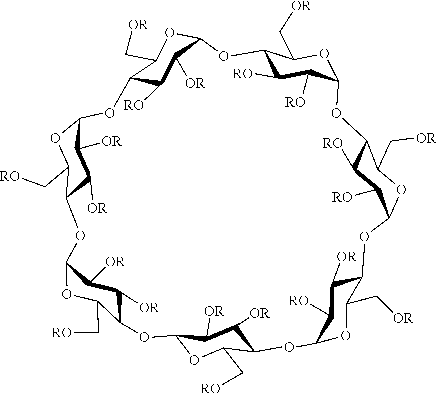

wherein the substituents R are each independently selected from the group consisting of H, alkyl, hydroxyalkyl, carboxyalkyl, alkylcarbonyl, carboxyalkoxyalkyl, —C₁₋₁₀-alkyl-SO₃H or a corresponding pharmaceutically acceptable salt thereof, alkylcarbonyloxyalkyl and alkoxycarbonylalkyl. In an embodiment, each alkyl moiety contains 1-10 carbon atoms, e.g. 1-6 carbon atoms, 2-4 carbon atoms or 3-4 carbon atoms. In an embodiment, the substituents R in Formula I are each independently selected from the group consisting of H, —CH₂CH(CH₃)OH, —(CH₂)₄SO₃Na, CH₃, glucosyl, hydroxyethyl and maltosyl. In an embodiment, the substituents R in Formula I are each independently selected from the group consisting of H, —CH₂CH(CH₃)OH, —(CH₂)₄SO₃Na and hydroxyethyl. In an especially advantageous embodiment, the substituents R in Formula I are each independently selected from the group consisting of H and —CH₂CH(CH₃)OH.

The degree of substitution of a cyclodextrin may be expressed in terms of average molar substitution, i.e. a measure of the average number of moles of all substituents per mole of glucose subunit. In an embodiment of the invention, a cyclodextrin of a formulation as described herein has a molar substitution (MS) ranging from 0.05 to 10, e.g. from 0.2 to 2, from 0.25 to 1 or from 0.5 to 0.8, e.g. about 0.65. In an embodiment of the invention, the cyclodextrin is 2-hydroxypropyl-β-cyclodextrin having an MS of 0.4 to 1.5 or 0.2 to 0.9, for example 0.3 to 0.8, 0.5 to 0.7, or 0.58 to 0.68. Alternatively, the degree of substitution of a cyclodextrin may be expressed in terms of the average number of substituents per cyclodextrin molecule. In an embodiment of the invention, a cyclodextrin of a formulation as described herein has, on average, between 4 and 8 substituents per cyclodextrin molecule, for example, on average, between 4 and 5, between 4 and 6, between 5 and 7 or between 6 and 8 substituents per cyclodextrin molecule.

In formulations of the invention, the pharmaceutically acceptable salt of dantrolene and the cyclodextrin (for example dantrolene sodium and 2-hydroxypropyl-β-cyclodextrin) are present in a molar ratio of 1:3 to 1:12. In preferred formulations, dantrolene and the cyclodextrin are present in a molar ratio of 1:5 to 1:10, for example a molar ratio of 1:6 to 1:9.5, 1:6.7 to 1:9.1, or 1:7 to 1:9, for example 1:8 to 1:9 or 1:8.3 to 1:8.6.

In formulations of the invention, the polyethylene glycol (PEG) is a solid PEG at room temperature (20° C.). For example, in formulations of embodiments of the invention, the PEG has an average molecular weight in the range 1500 to 6000. At room temperature (20° C.), these PEGs are solids. For example, the PEG has an average molecular weight in the range 2000 to 5000, for example 3000 to 4000. For example, the PEG may be PEG3000, PEG3350 or PEG4000 as defined in national pharmacopoeias, for example PEG3350. Further examples of suitable PEGs recognized in some national pharmacopoeias include Macrogols, for example Macrogol 4000. Optionally, the PEG used in formulations of the invention may comprise two or more different PEG compounds.

In certain embodiments of the formulations of the invention, the amount of PEG may be expressed as a w/w ratio relative to the amount of cyclodextrin. For example, the PEG may be present in an amount such that the w/w ratio of PEG to cyclodextrin is 1:2 to 1:50, for example 1:2 to 1:20. In an embodiment, the PEG is present in an amount such that the w/w ratio of PEG to cyclodextrin is 1:3 to 1:15, for example 1:5 to 1:12. Preferably, the w/w ratio of PEG to cyclodextrin is 1:8 to 1:10, more preferably 1:8.5 to 1:9. When the cyclodextrin is 2-hydroxypropyl-β-cyclodextrin, for example HPBCD with an MS of 0.3 to 0.8 or 0.5 to 0.7, the PEG (e.g. PEG having an average molecular weight in the range 3000-4000) is advantageously present in an amount such that the w/w ratio of PEG to cyclodextrin is 1:5 to 1:12 w/w relative to the amount of 2-hydroxypropyl-β-cyclodextrin, for example 1:8 to 1:10 or 1:8.5 to 1:9.

In certain embodiments of the formulations of the invention, the amount of PEG may be expressed as a w/w ratio relative to the amount of dantrolene. For example, the PEG (e.g. PEG having an average molecular weight in the range 3000-4000) may be present in an amount such that the w/w ratio of PEG to dantrolene is 1:0.1 to 1:10, for example 1:0.1 to 1:4 or 1:0.2 to 1:5. In an embodiment, the PEG is present in an amount such that the w/w ratio of PEG to cyclodextrin is 1:0.2 to 1:4, for example 1:0.2 to 1:3.5.

Alternatively, in certain embodiments of the formulations of the invention, the amount of PEG may be expressed as a concentration in mg/mL. For example, the PEG is present at a concentration of 0.5 to 50 mg/mL. For example, the PEG is present at a concentration of 2.5 to 50 mg/mL, for example 5 to 50 mg/mL. In an embodiment, the PEG is present at a concentration of 10 to 40 mg/mL, for example 15 to 30 mg/mL or 15 to 20 mg/mL. When the cyclodextrin is 2-hydroxypropyl-β-cyclodextrin, for example HPBCD with an MS of 0.3 to 0.8 or 0.5 to 0.7, the PEG (e.g. PEG having an average molecular weight in the range 3000-4000) is advantageously present at a concentration of 10 to 40 mg/mL, for example 15 to 30 or 15 to 20 mg/mL.

In certain embodiments of the formulations of the invention, it is beneficial to include certain further components such as pharmaceutical excipients and/or adjuvants, which are well known in the art. Excipients and/or adjuvants may be provided having specifications set out for example in the 2019 European Pharmacopeia (Ph. Eur.). Further examples of pharmaceutical excipients are set out in the Handbook of Pharmaceutical Excipients (9^{th} edition, 2020; Pharmaceutical Press (UK) and American Pharmaceutical Association (US)).

For example, the formulation of the invention may additionally include an antioxidant. An antioxidant may be included to enhance stability of the formulation of the invention by inhibiting deterioration due to oxidative processes. Exemplary antioxidants may include ascorbic acid. The formulation of the invention may additionally include a solubilising agent or crystallisation inhibitor. A solubilisation agent or crystallisation inhibitor may be included to maintain drug solubility and/or bioavailability. Exemplary solubilising agents or crystallisation inhibitors may include low molecular weight povidone, for example polyvinylpyrrolidone.

The formulation of the invention may additionally include an osmolality agent. An osmolality agent can be included to ensure that a solution prepared from the formulation of the invention when mixed with the required volume of solvent has the desired osmolality. For example, it can be beneficial for the solution to be iso-osmolar with the blood; that is to say that it has an osmolality of 270 to 300 mOsm/kg, particularly 285 to 290 mOsmol/kg. Exemplary osmolality agents include polyhydroxy alkanols having 2 to 10 carbon atoms, for example selected from the group consisting of mannitol, fructose, glucose, gluconolactone, gluconate, sucrose, lactose, trehalose, dextrose, dextran, hydroxyethyl starches and mixtures thereof. Further osmolality agents may be selected from the group consisting of glycine, gelatin, calcium gluconoglucoheptonate, potassium chloride, calcium chloride, sodium chloride and mixtures thereof. A combination of osmolality agents can be utilised to ensure that a solution prepared from the formulation of the invention when mixed with the required volume of solvent is isotonic with the blood.

The formulation of the invention may additionally include a pH adjuster. A pH adjuster can be included to ensure that a solution prepared from the formulation of the invention when mixed with the required volume of solvent has the desired pH, for example to improve stability, solubility, or suitability for the desired route of administration. Exemplary pH adjusters may include acids, bases, or buffers, for example citric acid, tartaric acid, hydrochloric acid, sodium hydroxide, sodium acetate, sodium citrate, sodium carbonate, sodium hydrogen carbonate, or calcium carbonate. As mentioned above, the present inventors have found that for most purposes no pH adjuster is needed for formulations of the invention. The formulations described herein achieve the desired solubility and other beneficial effects without the need to include further components to influence the pH. Nevertheless, in certain circumstances, a pH adjuster might be added to achieve a particular pH.

In preferred embodiments, the formulation of the invention is a dry formulation. It is beneficial to ensuring long term stability of a formulation of the invention for it to be dry. Dry formulations also have the benefits of being light (and thus easy to transport) and of low volume.

A dry formulation may be obtained in dry form by any suitable means. For example, a dry formulation may be a lyophilised formulation. A lyophilised formulation may for example be prepared using a method including the steps of: i) freezing; ii) an optional annealing step; iii) evacuation; (iv) primary drying; (v) secondary drying; (vi) stoppering with optional pre-aeration with N₂ and (vii) aeration with N₂ to atmospheric pressure. Alternatively, a dry formulation may be an air-dried formulation.

In an embodiment, the invention provides a lyophilised formulation which comprises dantrolene sodium and 2-hydroxypropyl-β-cyclodextrin in a molar ratio of 1:6 to 1:9.5 and which also comprises PEG with an average molecular weight in the range 3000 to 4000 and wherein, on average, each 2-hydroxypropyl-β-cyclodextrin molecule is substituted, on average by 4 to 6 2-hydroxypropyl groups. For example, such a formulation comprises dantrolene sodium hemiheptahydrate and 2-hydroxypropyl-β-cyclodextrin in a molar ratio of 1:8.3 to 1:8.6 and also comprises PEG3350, and wherein, on average, each 2-hydroxypropyl-β-cyclodextrin molecule is substituted by 5 2-hydroxypropyl groups.

As described above, the invention provides a liquid formulation prepared by dissolving a formulation as set out in the first claimed aspect of the invention in a pharmaceutically acceptable solvent. A pharmaceutically acceptable solvent for use in a liquid formulation can for example be water for injection Ph Eur.

As also described above, the invention provides an aqueous solution comprising a pharmaceutically acceptable salt of dantrolene and a cyclodextrin in a molar ratio of 1:3 to 1:12 and also comprising PEG with an average molecular weight in the range 1500 to 6000 at a concentration of 0.5 to 50 mg/mL. The invention also provides aqueous solutions which may be prepared from any of the dry formulations disclosed herein via the addition of a suitable pharmaceutically acceptable solvent.

In an embodiment, a liquid formulation according to the invention, for example an aqueous solution, comprises dantrolene, for example dantrolene sodium, at a concentration of from 0.4 to 10 mg/mL. Preferably, the dantrolene, for example dantrolene sodium, is present at a concentration of from 2 to 9 mg/mL, for example 4-8.5 mg/mL or 4-6.5 mg/mL. For example, the liquid formulation according to the invention may contain dantrolene, for example dantrolene sodium, at a concentration of about 4.5, 5.05, 5.4, or 6 mg/mL. The foregoing concentrations of dantrolene, for example dantrolene sodium, are expressed as anhydrous salt equivalent.

However, said concentrations may equally be expressed as dantrolene sodium 3.5 Mol hydrate equivalent (i.e. hemiheptahydrate equivalent), where 3.5 Mol hydrate denotes the presence of 3.5 water molecules per molecule of dantrolene (i.e. 3.5 moles of water per mole of dantrolene or dantrolene sodium). The terms “hemiheptahydrate equivalent” and “anhydrous salt equivalent” as used herein take into account the fact that anhydrous dantrolene sodium salt or dantrolene sodium hemiheptahydrate salt dissociate when they are dissolved in solution. Thus, for example, when solvent is added to 6 mg of dantrolene sodium hemiheptahydrate, to a total volume of 1 mL, this creates a solution comprising the equivalent of 6 mg/mL dantrolene sodium hemiheptahydrate.

It is possible to convert between concentrations of anhydrous dantrolene sodium and dantrolene sodium hemiheptahydrate by utilising the respective molecular masses of the two compounds. For example, a concentration of 4.55 mg/mL anhydrous dantrolene sodium (molecular mass 336.24 g/mol) converts to 5.4033 mg/mL 3.5 Mol hydrate (molecular mass 399.29 g/mol) and a concentration of 5.0525 mg/mL anhydrous dantrolene sodium converts to 6 mg/mL 3.5 Mol hydrate. In an embodiment, a liquid formulation according to the invention, for example an aqueous solution, comprises dantrolene, for example dantrolene sodium hemiheptahydrate, at a concentration of from 0.4 to 10 mg/mL (expressed as hemiheptahydrate equivalent). Preferably, the dantrolene, for example dantrolene sodium hemiheptahydrate, is present at a concentration of from 3 to 10 mg/mL, for example 4-8 mg/mL or 5-7 mg/mL, for example 6 mg/mL (all concentrations expressed as hemiheptahydrate equivalents).

Alternatively, the concentration of dantrolene may be expressed as mmol/mL (millimoles per mL). Therefore, in an embodiment, a liquid formulation according to the invention, for example an aqueous solution, comprises dantrolene at a concentration of from 0.001 to 0.05 mmol/mL, for example 0.005 to 0.025 mmol/mL or 0.01 to 0.020 mmol/mL. Preferably, the dantrolene is present at a concentration of from 0.012 to 0.016 mmol/mL, for example 0.013 mmol/mL or 0.015 mmol/mL. As can readily be calculated, a liquid formulation according to the invention comprising dantrolene sodium at a concentration of 6 mg/mL hemiheptahydrate equivalent (or 5.0525 mg/mL anhydrous equivalent) results in a concentration of 0.015 mmol/mL dantrolene.

In an embodiment, the liquid formulation according to the invention comprises a 2-hydroxypropyl-β-cyclodextrin present at a concentration of 100 to 300 mg/mL. Preferably, the 2-hydroxypropyl-β-cyclodextrin is present at a concentration of 100 to 200 mg/mL, for example 120 to 180 mg/mL or 150 to 180 mg/mL. More preferably, the concentration of the 2-hydroxypropyl-β-cyclodextrin is 176.5 mg/mL. Most preferably, the 2-hydroxypropyl-β-cyclodextrin is present at a concentration of 140 to 170 mg/mL or 150 to 160 mg/mL, for example 156.2 mg/mL. Preferably, the 2-hydroxypropyl-β-cyclodextrin is present at a concentration of 100-200 mg/mL, for example 120-190 mg/mL, for example 150-185 mg/mL, for example 150-180 mg/mL, or for example 175-177 mg/mL. Preferably, the concentration of the 2-hydroxypropyl-β-cyclodextrin is 176.5 mg/mL. Most preferably, the 2-hydroxypropyl-β-cyclodextrin is present at a concentration of 140-170 mg/mL or 150-160 mg/mL, for example 156.2 mg/mL. As described herein, the pharmaceutically acceptable salt of dantrolene and the cyclodextrin are present in a formulation as described herein in particular molar ratio ranges, e.g. within the range 1:3 to 1:12, 1:5 to 1:10, 1:6 to 1:9.5, 1:6.7 to 1:9.1, 1:7 to 1:9, 1:8 to 1:9 or 1:8.3 to 1:8.6.

In an embodiment, the liquid formulation according to the invention comprises a PEG, for example PEG3350, present at a concentration of 0.5-50 mg/mL, for example 5-45 mg/mL, for example 10-40 mg/ml, or for example 10, 20, 30, or 40 mg/mL. Preferably, the PEG, for example PEG3350, is present at a concentration of 5-25 mg/mL, for example 10-25 mg/mL or 15-25 mg/mL, suitably from 15 to 20 mg/ml. Preferably, the PEG, for example PEG3350, is present at a concentration of 17.7 mg/mL or 20 mg/mL.

In an embodiment, a liquid formulation according to the invention, for example an aqueous solution, comprises: dantrolene, for example dantrolene sodium, for example dantrolene sodium hemiheptahydrate, present at a concentration of 0.4-10 mg/mL (expressed as dantrolene sodium hemiheptahydrate equivalent); a cyclodextrin, for example 2-hydroxypropyl-β-cyclodextrin present at a concentration of 100-300 mg/mL; and a PEG, for example PEG3350, present at a concentration of 0.5-50 mg/mL. Preferably, the liquid formulation according to the invention, for example an aqueous solution, comprises: dantrolene, for example dantrolene sodium, for example dantrolene sodium hemiheptahydrate, present at a concentration of 2-9 mg/ml (dantrolene sodium hemiheptahydrate equivalent); a cyclodextrin, for example 2-hydroxypropyl-β-cyclodextrin present at a concentration of 100-200 mg/mL; and a PEG, for example PEG3350, present at a concentration of 5-25 mg/mL. More preferably, the liquid formulation according to the invention, for example an aqueous solution, comprises: dantrolene, for example dantrolene sodium, for example dantrolene sodium hemiheptahydrate, present at a concentration of 4-6.5 mg/mL (dantrolene sodium hemiheptahydrate equivalent); a cyclodextrin, for example 2-hydroxypropyl-β-cyclodextrin present at a concentration of 120-190 mg/mL; and a PEG, for example PEG3350, present at a concentration of 15-25 mg/mL. For example, the liquid formulation according to the invention, for example an aqueous solution, may comprise: dantrolene, for example dantrolene sodium, for example dantrolene sodium hemiheptahydrate, present at a concentration of 6 mg/mL (dantrolene sodium hemiheptahydrate equivalent); a cyclodextrin, for example 2-hydroxypropyl-β-cyclodextrin present at a concentration of 176.5 mg/mL; and a PEG, for example PEG3350, present at a concentration of 20 mg/mL. For example, the liquid formulation according to the invention, for example an aqueous solution, may comprise: dantrolene, for example dantrolene sodium, present at a concentration of 5.3 mg/mL (dantrolene sodium hemiheptahydrate equivalent); a cyclodextrin, for example 2-hydroxypropyl-β-cyclodextrin present at a concentration of 156.2 mg/mL; and a PEG, for example PEG3350, present at a concentration of 17.7 mg/mL.

A liquid formulation according to an embodiment of the invention advantageously has a pH that is greater than 7, for example a pH of 8 to 11, for example a pH of 8.8 to 11, for example a pH of 8.8 to 10 or for example a pH of 9 to 10. The pH of the liquid formulation according to the invention may preferably be 8 to 9.5 or 9 to 9.5, or may more preferably be 9.2 to 9.5.

The invention also provides a kit comprising: a first container, e.g. a vial as described herein, which contains a formulation of an embodiment of the invention as described herein; and a second vessel containing a diluent, i.e. pharmaceutically acceptable solvent. Preferably, the formulation of the invention is a dry formulation, for example a lyophilized formulation. Examples of pharmaceutically acceptable solvents include those described herein as being suitable for use in the invention. Preferably, the first and second containers of the kit of the invention are vials, as disclosed herein.

Kits of the invention find use in the treatment of malignant hyperthermia. Kits of the invention may also find use in the treatment of chronic severe spasticity or neuroleptic malignant syndrome. Alternatively, kits of the invention may also find use in the treatment of conditions including acute radiation syndrome in hematopoietic syndrome patients exposed to high doses of radiation, psychostimulant drug-induced toxicity (MDMA and Methamphetamine Intoxication), exertional heat stroke, cerebral concussion and other forms of traumatic brain injury, nerve-agent induced brain damage, and/or coronavirus infection (COVID-19).

The invention further provides a formulation, a liquid formulation (for example a liquid formulation prepared by dissolving a dry formulation, such as a lyophilized formulation, as described herein, in a pharmaceutically acceptable solvent), a vial, a kit or an aqueous solution as described herein, for use as a medicament.

The invention also provides a formulation, a liquid formulation (for example a liquid formulation prepared by dissolving a dry formulation, such as a lyophilized formulation, as described herein, in a pharmaceutically acceptable solvent), a vial, a kit or an aqueous solution as described herein, for the manufacture of a medicament for the treatment of malignant hyperthermia.

The invention also provides a formulation, a liquid formulation (for example a liquid formulation prepared by dissolving a dry formulation, e.g. a lyophilized formulation, as described herein in a pharmaceutically acceptable solvent), a vial, a kit or an aqueous solution as described herein, for use in the treatment of severe spasticity or neuroleptic malignant syndrome.

The invention further provides a formulation, a liquid formulation (for example a liquid formulation prepared by dissolving a dry formulation, e.g. a lyophilized formulation, as described herein in a pharmaceutically acceptable solvent), a vial, a kit or an aqueous solution as described herein, for use in the treatment of acute radiation syndrome in hematopoietic syndrome patients exposed to high doses of radiation, psychostimulant drug-induced toxicity (MDMA and Methamphetamine intoxication), exertional heat stroke, cerebral concussion and other forms of traumatic brain injury, nerve-agent induced brain damage, or coronavirus infection (COVID-19).

The invention also provides a method for treating malignant hyperthermia in a subject in need thereof, which comprises administering to said subject a pharmaceutically effective amount of a formulation, a liquid formulation (for example a liquid formulation prepared by dissolving a dry formulation, e.g. a lyophilized formulation, as described herein in a pharmaceutically acceptable solvent), or an aqueous solution as described herein.

The invention further provides a method for treating chronic severe spasticity or neuroleptic malignant syndrome in a subject in need thereof, which comprises administering to said subject a pharmaceutically effective amount of a formulation, a liquid formulation (for example a liquid formulation prepared by dissolving a dry formulation, e.g. a lyophilized formulation, as described herein in a pharmaceutically acceptable solvent), or an aqueous solution as described herein.

The invention further provides a method for treating: acute radiation syndrome in hematopoietic syndrome patients exposed to high doses of radiation, psychostimulant drug-induced toxicity (MDMA and Methamphetamine intoxication), exertional heat stroke, cerebral concussion and other forms of traumatic brain injury, nerve-agent induced brain damage, or coronavirus infection (COVID-19); in a subject in need thereof, which comprises administering to said subject a pharmaceutically effective amount of a formulation, a liquid formulation (for example a liquid formulation prepared by dissolving a dry formulation, e.g. a lyophilized formulation, as described herein in a pharmaceutically acceptable solvent), or an aqueous solution as described herein

The invention also provides a method of manufacturing a formulation, a liquid formulation, a kit or an aqueous solution as described herein. A formulation of an embodiment of the invention as described herein may be manufactured by a method comprising the steps of: (i) providing a cyclodextrin, e.g. a 2-hydroxypropyl-β-cyclodextrin, stock solution in a pharmaceutically acceptable solvent; (ii) adding a PEG (e.g. PEG3000, PEG3350 or PEG4000) and adding dantrolene (e.g. dantrolene sodium) to the cyclodextrin stock solution; (iii) mixing of the solution to allow dissolution of the dantrolene and (iv) optionally adding further cyclodextrin stock solution if required to achieve final weight. In the context of said step (ii), the PEG and the dantrolene may be added to the stock solution in either order or simultaneously. Said method may further comprise the steps of: (v) filtration; (vi) filling of vials; (vii) drying of the formulation, e.g. by lyophilisation, optionally including an annealing step and (viii) closure of vials. Said step of lyophilisation may comprise the steps of: (a) freezing; (b) an optional annealing step; (c) evacuation; (d) primary drying; (e) secondary drying; (f) stoppering with optional pre-aeration with N₂ and (g) aeration with N₂ to atmospheric pressure.

The invention further provides a formulation, a liquid formulation, or an aqueous solution as described herein, obtainable by a process comprising the steps of: (i) providing a cyclodextrin, e.g. a 2-hydroxypropyl-β-cyclodextrin, stock solution in a pharmaceutically acceptable solvent; (ii) adding a PEG (e.g. PEG3000, PEG3350 or PEG4000) and adding dantrolene (e.g. dantrolene sodium) to the cyclodextrin stock solution; (iii) mixing of the solution to allow dissolution of the dantrolene and (iv) optionally adding further cyclodextrin stock solution if required to achieve final weight. In the context of said step (ii), the PEG and the dantrolene may be added to the stock solution in either order or simultaneously.

The invention further provides a dry formulation as described herein, obtainable by a process comprising the steps of: (i) providing a cyclodextrin, e.g. a 2-hydroxypropyl-β-cyclodextrin, stock solution in a pharmaceutically acceptable solvent; (ii) adding a PEG (e.g. PEG3000, PEG3350 or PEG4000) and adding dantrolene (e.g. dantrolene sodium) to the cyclodextrin stock solution; (iii) mixing of the solution to allow dissolution of the dantrolene; (iv) optionally adding further cyclodextrin stock solution if required to achieve final weight and (v) drying the resultant solution, e.g. by lyophilisation, to obtain said dry formulation. In the context of said step (ii), the PEG and the dantrolene may be added to the stock solution in either order or simultaneously.

The invention further provides a dry formulation as described herein, comprising 100-120 mg dantrolene sodium, 3000-4000 mg 2-hydroxypropyl-β-cyclodextrin and 350-450 mg PEG3350.

As described above, the invention further provides a vial comprising: (i) 101 mg anhydrous dantrolene sodium (equivalent to 120 mg dantrolene sodium hemiheptahydrate), which may also be expressed as 0.3 mmol dantrolene; (ii) 3.53 g 2-hydroxypropyl-β-cyclodextrin and (iii) 400 mg PEG3350.

The invention further provides a vial comprising: (i) 0.3 mmol dantrolene; (ii) 3.53 g 2-hydroxypropyl-β-cyclodextrin and (iii) 400 mg PEG3350.

A further object of the present invention is a method for the preparation of a liquid formulation or an aqueous composition as described herein. The method comprises the steps of dissolving a dry formulation as described herein in an aqueous diluent.

The amount of the formulation according to the invention which is required to achieve a therapeutic effect will vary with the particular route of administration and the characteristics of the subject under treatment (for example the age, weight, sex, or other concurrent medical conditions) and can be readily determined and administered by an ordinarily skilled physician. Preferably, the recommended dosage regime ranges from 1-10 mg dantrolene sodium per kg body weight, for example 10 mg/kg. In another embodiment, the dosage regime ranges from 1.5 mg/kg to 3.5 mg/kg, for example 2.5 mg/kg. An ordinarily skilled physician can calculate the number of vials required to obtain a particular dosage to be administered to a subject.

The formulations of the invention are preferably administered parenterally, more preferably intravenously. Therefore, in an embodiment, the liquid formulations according to the invention are preferably administered parenterally, more preferably intravenously.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

### EXAMPLES

### Example 1: Effect of Adding 2-hydroxypropyl-β-cyclodextrin (HPBCD) to Dantrolene Sodium Solutions

This study was performed to determine the impact of HPBCD on the solubility of dantrolene Sodium. An initial comparison of different HPBCDs was carried out using HP5 and HP7 substituted variants (Cavitron™ W7, Ashland, Inc.), which have a typical degree of substitution (average number of hydroxypropyl groups per cyclodextrin molecule) of 4.1-5.1 and 6.0-8.0 respectively. (Equivalent products, such as Kleptose® HPB (Roquette), which is a low endotoxin burden (<10 IU/g) HP5 substituted β-cyclodextrin with typical molar substitution (i.e. per anhydro glucose subunit) of 0.65 (range 0.58 to 0.68), are also available for commercial scale manufacturing.)

The solubility achieved for both HP5 and HP7 variants was 8.5 mg/mL dantrolene sodium hemiheptahydrate equivalent in 9 Mol equivalents of HPBCD, using purified water as the solvent. Due to the larger molecular weight of the HP7 variant, more weight of excipient was required to achieve the same molar equivalent concentration compared with the HP5 variant. Overall, HP5 substituted β-cyclodextrin was preferred and it was therefore used for subsequent test formulations.

### 1A: Effect of Particle Size on Dantrolene Sodium Solubility in HPBCD Solution

Further analysis was performed to determine the maximum possible concentration of dantrolene sodium, at different particle sizes, in water only solutions of HP5 substituted β-cyclodextrin. Commercially available samples of dantrolene sodium having particle sizes of <2 μm and <63 μm were tested. The following experimental conditions were used for all test formulations: HPBCD concentration, 7 molar equivalent (expressed relative to the dantrolene sodium hemiheptahydrate salt). Results are shown in Table 1 below; DNa denotes dantrolene sodium.

TABLE 1
Dantrolene sodium solubility in water only solutions of HPBCD.

| DNa concentration | DNa concentration | | | |
|---|---|---|---|---|
| (Anhydrous salt | (Hemiheptahydrate | DNa particle | | |
| equivalent) (mg/mL) | equivalent) (mg/mL) | size (μm) | | Result (assessed by visual inspection) |
| 4.5 | 5.33 | <2 | μm | Complete solution |
| 4.46 | 5.16 | <2 | μm | Complete solution |
| 4.55 | 5.36 | <2 | μm | Complete solution |
| 4.6 | 5.40 | <2 | μm | Slightly Hazy Solution (Incomplete) |
| 4.65 | 5.47 | <2 | μm | Hazy Solution (Incomplete) |
| 4.75 | 5.58 | <2 | μm | Hazy Solution (Incomplete) |
| 5.0 | 5.95 | <2 | μm | Hazy Solution (Incomplete) |
| 4.5 | 5.35 | <63 | μm | Complete solution |
| 4.75 | 5.66 | <63 | μm | Hazy Solution (Incomplete) |

For the <2 μm particle size, the maximum concentration of dantrolene sodium that could be dissolved under these conditions was 4.55 mg/mL as anhydrous salt equivalent (5.36 mg/mL as hemiheptahydrate equivalent). Solubility for the <63 μm particle size was similar, at 4.5 mg/mL, as anhydrous salt equivalent (5.35 mg/mL as hemiheptahydrate equivalent).

Both these results and the results of the initial comparison between HP5 and HP7 variants demonstrate that adding HPBCD increases dantrolene sodium solubility compared with existing commercial formulations; for example, the DANTRIUM® IV formulation (Norgine Pharmaceuticals, Uxbridge, UK) achieves 0.33 mg/mL solubility (as dantrolene sodium hemiheptahydrate equivalent). Furthermore, the similarity in solubility across both small (2 micron) and large (63 micron) particle sizes when combined with HPBCD is notable, given the current requirement for micronisation of dantrolene sodium to ≤2 μm diameter for DANTRIUM® IV, in order to permit sufficient solubility.

### 1B: Impact of Parenteral-Compatible Co-Solvents on Dantrolene Solubility in HPBCD Solution

This experiment was performed to determine the effects of different parenteral-compatible alcohol- and ketone-based co-solvents on dantrolene sodium solubility, in a 7 Mol equivalent HP5 substituted HPBCD solution. The results are shown in Table 2 below; DNa denotes dantrolene sodium.

TABLE 2
Dantrolene sodium solubility with HPBCD and solvent addition

| DNa (Anhydrous | | | | |
|---|---|---|---|---|
| salt equivalent) | | Solvent | Solvent | Result (assessed by |
| (mg/mL) | Solvent | Purity (%) | concentration (%) | visual inspection) |
| 5.0 | Benzyl Alcohol | 99.8 | 2 | Large Excess |
| 5.0 | Ethanol Abs. | 99.99 | 20 | Large Excess |
| 5.0 | Tert-Butanol | 99.5 | 20 | Large Excess |
| 5.0 | Tert-Butanol | 99.5 | 10 | Large Excess |
| 4.75 | Tert-Butanol | 99.5 | 5 | Large Excess |
| 4.5 | Tert-Butanol | 99.5 | 5 | Large Excess |
| 4.5 | Propylene Glycol | 99.5 | 5 | Moderate Excess |
| 4.5 | Polyethylene Glycol 300 (PEG-300) | 100 | 5 | Hazy - Small Excess |
| 4.5 | Polyethylene Glycol 400 (PEG-400) | 100 | 5 | Hazy - Small Excess |
| 4.5 | Glycerol | 99.5 | 5.18 | Hazy - Small Excess |
| 4.5 | Benzyl Alcohol | 99.8 | 1 | Moderate Excess |
| 4.5 | Ethanol Abs. | 99.99 | 1 | Hazy - Small Excess |
| 4.5 | Tetrahydrofuran | 99.9 | 2 | Large Excess |
| 4.5 | N,N-Dimethyl-formamide | 99.8 | 2 | Hazy - Small Excess |
| 4.55 | N-Methyl-pyrrolidone | 99.92 | 2, 4, 6 | Hazy - Small Excess |

These results show that the addition of parenteral-compatible alcohol- or ketone-based solvents did not improve dantrolene Sodium solubility in the presence of HPBCD; rather, all 15 of the solvents tested, including PEG 300 and PEG 400, reduced dantrolene solubility.

1C: Comparison of a Dantrolene-HPBCD Formulation with an Existing Commercial Dantrolene Formulation

Table 3 below compares an existing commercial dantrolene formulation, DANTRIUM IV®, with a dantrolene/HP5 substituted HPBCD formulation. DNa denotes dantrolene sodium.

TABLE 3
Properties of dantrolene sodium formulations
with and without HPBCD

| Feature/ | DANTRIUM IV ® | Dantrolene/HPBCD |
|---|---|---|
| Property | (commercial formulation) | (test formulation) |
| Ingredients | 18.6 mg anhydrous DNa | 101 mg anhydrous DNa |
| (per vial) | (equivalent to 20 mg | (equivalent to 120 mg |
| | DNa 3.5 Mol hydrate) | DNa 3.5 Mol hydrate) |
| | 3.0 g mannitol | 3530 mg 2-hydroxypropyl |
| | NaOH to achieve pH 9.5 | β-cyclodextrin (molar |
| | | substitution range of |
| | | 0.58-0.68) |
| | | 400 mg PEG 3350 |
| DNa particle | ≤2 μm needed to | Larger particle sizes |
| size needed | maintain dantrolene | can be used with little |
| | concentration in | to no impact on |
| | the reconstituted | solubility, e.g. 40 μm |
| | solution | |
| Concentration | 0.33 mg/mL DNa | 5.3 mg/mL DNa |
| of DNa after | hemiheptahydrate | hemiheptahydrate |
| reconstitution | equivalent | equivalent |
| Volume of | 60 mL water for | 20 mL water for |
| solvent for | injection | injection |
| reconstitution | | |
| (per vial) | | |
| Vial size | 75 mL | 50 mL |
| Buffering | Yes | No: pH of reconstituted |
| requirement | | solution is naturally |
| | | alkaline (pH consistently |
| | | between 9.2 and 9.5) and |
| | | remains stable without |
| | | need for alteration and/ |
| | | or buffering |
| Reconstitution | 15 min (approx.) | ≤90 seconds per vial |
| time | per vial | |

As shown in Table 3, the formulation with HPBCD offers the following advantages compared with an existing dantrolene formulation: higher concentration of dantrolene when reconstituted; lower reconstitution volume; increased speed of reconstitution; smaller vial size required; the ability to use dantrolene particles of larger size, without the need for micronisation to 2 μm; and elimination of the need for buffering.

### Example 2—Effect of Adding Antifoaming Agents

This experiment was performed to evaluate the effect of adding antifoaming agents to a dantrolene-HPBCD formulation (6 mg/mL dantrolene sodium API+17.65% w/v (176.5 mg/mL equivalent) HPBCD (molar substitution range of 0.58-0.68), both ingredients obtained from commercial sources). Initial testing of dimethicone at 2% w/v (20 mg/mL equivalent) yielded poor results: the dimethicone did not dissolve fully and remained stratified over the solution.

Commercially available PEG 3350 was tested as an alternative anti-foaming agent, using the following preparation process:
(i) Preparation of 17.65% w/v HPBCD stock solution in water for injection;
(ii) Addition of 2% w/v PEG 3350 to the 17.65% w/v HPBCD solution (90% of final volume);
(iii) Heating of the resulting PEG 3350/HPBCD solution to 30±5° C. (O₂ concentration reduced below 2 ppm by N₂-insufflation);
(iv) Dispensing of the dantrolene sodium API into the pre-heated PEG 3350/HPBCD solution;
(v) Mixing of the solution at 30ºC for at least 30 minutes (until complete dissolution of the dantrolene sodium API);
(vi) Solution cooled to room temperature (15-25° C.), oxygen concentration reduced by N₂-insufflation, and solution brought to final weight using 17.65% w/v HPBCD stock solution;
(vii) Filtration of solution using 0.22 μm PVDF filter;
(viii) Filling and pre-stoppering of vials;
(ix) Lyophilisation (including the steps of: i) freezing; ii) an optional annealing step; iii) evacuation; (iv) primary drying; (v) secondary drying; (vi) pre-aeration with N₂ and stoppering and (vii) aeration with N₂ to atmospheric pressure and final closure of vials);
(x) Inspection and labelling of vials.

Addition of PEG 3350 resulted in a clear and homogeneous solution. Upon shaking (after reconstitution of the lyophilised formulation with water for injection), the PEG 3350 formulation generated less foam that cleared more quickly (as determined by visual inspection) compared to the formulation without antifoaming agent, which was prepared using the same method as above but omitting step (ii). Surprisingly, given that other solvents, including lower molecular weight PEGs (PEG 300/400), reduced dantrolene solubility when added to the solution (Example 1B), PEG 3350 did not decrease the solubility of the dantrolene sodium. (Similar results were also obtained using a higher concentration of PEG 3350 (4% w/v; 40 mg/mL equivalent)). These results identify PEG 3350 as a surprisingly effective anti-foaming agent for the dantrolene formulation which does not reduce the solubility of the API.

### Example 3—Exemplary Dantrolene Formulation

An example of a dantrolene formulation suitable for clinical use is described below.

The formulation is a lyophilised preparation containing commercially available dantrolene sodium hemiheptahydrate (API particle size 40 μm), 2-hydroxypropyl-β-cyclodextrin and PEG3350 prepared according to the method detailed in Example 2. The preparation is packaged in 50 mL vials, with each vial containing: 101 mg anhydrous dantrolene sodium (equivalent to 120 mg dantrolene sodium hemiheptahydrate), which may also be expressed as 0.3 mmol dantrolene; 3530 mg 2-hydroxypropyl-β-cyclodextrin; and 400 mg PEG3350. Reconstitution of the preparation in 20 mL water for injection yields concentrations of 5.3 mg/mL for dantrolene sodium hemiheptahydrate equivalent, 156.2 mg/mL for 2-hydroxypropyl-β-cyclodextrin, and 17.7 mg/mL for PEG3350 in a total volume of 22.6 mL. The reconstitution time is ≤90 seconds. The reconstituted solution achieves a stable pH between 9.2 and 9.5, and does not require pH buffering.

### Example 4—Effect of PEG on Foaming in Comparative Dantrolene Solutions

An exemplary formulation of the invention (Formulation 5) was compared against a number of prior art formulations (Formulations 1 to 4) to determine the effect of PEG on the solutions.

Formulations 1-5 were made as detailed below to give a total volume of 20 mL for each solution. Moreover, modified versions of Formulations 1-4 were also made as detailed below to give a total volume of 20 mL for each solution, with the addition of PEG 3350 (Macrogol 3350) to give a weight ratio of PEG to dantrolene of 1:3.333. All solutions were shaken for 3 minutes by hand. The height of the foam in each of the solutions was measured as was the time it took for the foam to subside. A visual inspection of the solutions was made and any issues with solubility were noted. Results are shown in Table 4 below.

TABLE 4
Impact of PEG on foam height and time to subside upon shaking

| | | Time for | |
|---|---|---|---|
| | Foam | foam to | |
| | height | subside | |
| Formulation | (mm) | (seconds) | Visual Inspection |
| 1 + PEG | 11 | 34 | Before filtration solution remained |
| | | | cloudy, clear solution was obtained |
| | | | after filtration |
| 1 − PEG | 5 | 94 | Before filtration solution remained |
| | | | cloudy, clear solution was obtained |
| | | | after filtration |
| 2 + PEG | 9 | 45 | — |
| 2 − PEG | 26 | 128 | — |
| 3 + PEG | 7 | 14 | — |
| 3 − PEG | 21 | 960 | Foam was still present after 16 |
| | | | minutes |
| 4 + PEG | 7 | 47 | Before filtration solution remained |
| | | | cloudy, clear solution was obtained |
| | | | after filtration |
| 4 − PEG | 16 | 279 | Before filtration solution remained |
| | | | cloudy, clear solution was obtained |
| | | | after filtration |
| 5 | 10 | 52 | — |

These results show that the addition of PEG to the prior art dantrolene Formulations 1-4 reduced the time taken for foaming to subside following shaking. In addition, PEG also reduced the height of the foam in the vial for prior art Formulations 2, 3, and 4. Furthermore, a comparison of Formulation 5 with Formulations 1 to 4 (in the absence of PEG) shows that the time taken for foam to subside in Formulation 5 was much reduced when compared with prior art Formulations 1 to 4 (which do not contain PEG), i.e. the time required for preparing a solution of dantrolene suitable for injection according to Formulation 5 would be shorter when compared with prior art formulations.

Formulation 1 was prepared according to Chen et al. (Journal of Pharmaceutical and Biomedical Analysis 135 (2017) 153-159) as described in section 2.2 up to but not including the freeze-drying steps. Formulation 2 was prepared according to WO 2017/067980, Example 4. Formulation 3 was prepared according to WO 2017/067980, Solution 2. Formulation 4 was prepared according to WO 2018/146187, Example 2 Formulation 5 is an exemplary formulation of the invention prepared according to the method detailed in Example 2, process steps (i) to (vii).

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A formulation comprising dantrolene, or a pharmaceutically acceptable salt thereof, and a cyclodextrin in a molar ratio of 1:3 to 1:12 and also comprising polyethylene glycol (PEG) with an average molecular weight in the range 1500 to 6000.

2. The formulation as claimed in claim 1, wherein said pharmaceutically acceptable salt of dantrolene is the sodium salt, optionally the sodium hemiheptahydrate salt or the anhydrous sodium salt.

3. The formulation as claimed in claim 1, wherein said cyclodextrin contains between 5 and 10 glucose subunits and wherein said glucose subunits of said cyclodextrin are optionally substituted with a C₁₋₆ alkyl group, which is itself optionally substituted with a hydroxyl group or a sulfo group.

4. The formulation as claimed in claim 3, wherein said glucose subunits of said cyclodextrin are substituted with a C₂₋₄ hydroxyalkyl group, optionally a 2-hydroxypropyl group.

5. (canceled)

6. The formulation as claimed in claim 3, wherein on average, each cyclodextrin molecule contains between 4 and 8 substituents, preferably 5 substituents.

7. The formulation as claimed in claim 1, wherein said cyclodextrin contains 6 glucose subunits (α-cyclodextrin), 7 glucose subunits (β-cyclodextrin) or 8 glucose subunits (γ-cyclodextrin), optionally wherein said cyclodextrin contains 7 glucose subunits (β-cyclodextrin).

8. (canceled)

9. The formulation as claimed in claim 1, wherein said cyclodextrin is a β-cyclodextrin of Formula I: 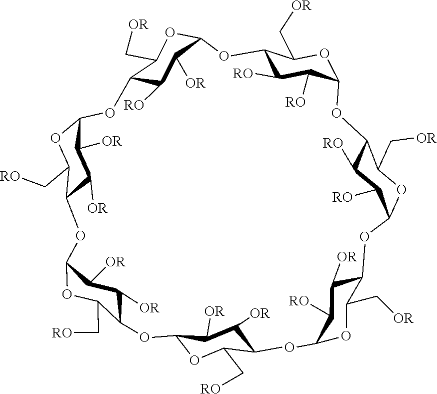
wherein each R substituent is independently selected from the group consisting of H, —CH₃, —CH₂CH(CH₃)OH, —(CH₂)₄SO₃Na and hydroxyethyl.

10. The formulation as claimed in claim 9, wherein each R substituent is independently selected from the group consisting of H and —CH₂CH(CH₃)OH.

11. The formulation as claimed claim 1, wherein said dantrolene or a pharmaceutically acceptable salt thereof and said cyclodextrin are present in a molar ratio of 1:5 to 1:10, optionally 1:6 to 1:9.5.

12. (canceled)

13. (canceled)

14. The formulation as claimed in claim 1, wherein said PEG has an average molecular weight in the range 3000 to 4000, optionally wherein said PEG is PEG3000, PEG3350 or PEG4000.

15. (canceled)

16. (canceled)

17. The formulation as claimed in claim 1, wherein said PEG is present in an amount such that the w/w ratio of PEG to cyclodextrin is 1:3 to 1:15, optionally 1:5 to 1:12.

18. (canceled)

19. (canceled)

20. The formulation as claimed in claim 1, which comprises dantrolene sodium hemiheptahydrate and 2-hydroxypropyl-β-cyclodextrin in a molar ratio between 1:8.3 to 1:8.6 and which also comprises PEG3350 in an amount such that the w/w ratio of PEG3350 to 2-hydroxypropyl-β-cyclodextrin is between 1:8.5 and 1:9, and wherein, on average, each 2-hydroxypropyl-β-cyclodextrin molecule is substituted by 5 hydroxypropyl groups.

21. (canceled)

22. (canceled)

23. The formulation as claimed in claim 1, which is a dry formulation.

24. The dry formulation as claimed in claim 23, which is a lyophilised formulation.

25. A dry formulation comprising 100-130 mg dantrolene sodium, 3000-4000 mg 2-hydroxypropyl-β-cyclodextrin and 350-450 mg PEG3350.

26. A liquid formulation prepared by dissolving a dry formulation as claimed in claim 23 in a pharmaceutically acceptable solvent.

27. An aqueous solution comprising dantrolene or a pharmaceutically acceptable salt thereof and a cyclodextrin in a molar ratio of 1:3 to 1:12 and also comprising PEG with an average molecular weight in the range 1500 to 6000.

28. The aqueous solution as claimed in claim 27, wherein the pharmaceutically acceptable salt of dantrolene, the cyclodextrin and the PEG are as defined in claim 1.

29. The liquid formulation as claimed in claim 26, or the aqueous solution as claimed in claim 27, wherein the pH of said formulation or said solution is greater than 7.0, optionally wherein the pH of said formulation is between 8.0 and 10.0.

30. (canceled)

31. (canceled)

32. (canceled)

33. (canceled)

34. A method for treating malignant hyperthermia in a subject in need thereof, wherein the method comprises administering to said subject pharmaceutically effective amount of a formulation as claimed in claim 1, an aqueous solution as claimed in claim 17 or a liquid formulation as claimed in claim 19.

35-43. (canceled)
